SEQUENCE LISTING (17 sequences)

SEQ ID NO: 1 - DNA, 21 bp, Artificial Sequence
  Description of Artificial Sequence Synthetic primer
ccagagagatctttgtgaagg

SEQ ID NO: 2 - DNA, 87 bp, Artificial Sequence
  Description of Artificial Sequence Synthetic primer
gcgcgctgtacatattatgatatggatacaacgtatgcaatggccaatagccaatattggcgcgcctcaccgtccttgacacgaagc

SEQ ID NO: 3 - DNA, 37 bp, Artificial Sequence
  Description of Artificial Sequence Synthetic primer
cgtataggcgcgcctactgagtcattagggactttcc

SEQ ID NO: 4 - DNA, 35 bp, Artificial Sequence
  Description of Artificial Sequence Synthetic primer
gcatcgaagcttctgcgttctacggtggtcagacc

SEQ ID NO: 5 - DNA, 39 bp, Artificial Sequence
  Description of Artificial Sequence Synthetic primer
gatcggcgcgcctaagctactgagtcattagggactttc

SEQ ID NO: 6 - DNA, 29 bp, Artificial Sequence
  Description of Artificial Sequence Synthetic primer
gatccctgaggctgcgttctacggtggtc

SEQ ID NO: 7 - DNA, 29 bp, Artificial Sequence
  Description of Artificial Sequence Synthetic primer
gatccctcaggacctccatagaagacacc

SEQ ID NO: 8 - DNA, 23 bp, Artificial Sequence
  Description of Artificial Sequence Synthetic primer
gatcaagcttcgtgtcaaggacg

SEQ ID NO: 9 - DNA, 34 bp, Artificial Sequence
  Description of Artificial Sequence Synthetic primer
gatcgctagcggccgctgaggcgcgcctactgag

SEQ ID NO: 10 - DNA, 949 bp, Artificial Sequence
  Description of Artificial Sequence Synthetic polynucleotide
ccagagagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctctagcttcgtgtcaaggacggtgaggcgcgccaatattggctattggccattgcatacgttgtatccatatcataatatgtacagcgcgc

SEQ ID NO: 11 - DNA, 552 bp, Artificial Sequence
  Description of Artificial Sequence Synthetic polynucleotide
cgtataggcgcgcctactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagaagcttcgatgc

SEQ ID NO: 12 - DNA, 555 bp, Artificial Sequence
  Description of Artificial Sequence Synthetic polynucleotide
gatcggcgcgcctaagctactgagtcattagggactttcaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagcctcagggatcga

SEQ ID NO: 13 - DNA, 951 bp, Artificial Sequence
  Description of Artificial Sequence Synthetic polynucleotide
gatccctcaggacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgatc

SEQ ID NO: 14 - DNA, 1481 bp, Artificial Sequence
  Description of Artificial Sequence Synthetic polynucleotide
ggcgcgcctaagctactgagtcattagggactttcaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagcctcaggacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagctt

SEQ ID NO: 15 - DNA, 565 bp, Artificial Sequence
  Description of Artificial Sequence Synthetic polynucleotide
gatcgctagcggccgctgaggcgcgcctactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagaagcttcgatgc

SEQ ID NO: 16 - DNA, 565 bp, Artificial Sequence
  Description of Artificial Sequence Synthetic polynucleotide
gatcgctagcggccgctgaggcgcgcctactgagtcattagggactttcaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagcctcagggatcga

SEQ ID NO: 17 - DNA, 1487 bp, Artificial Sequence
  Description of Artificial Sequence Synthetic polynucleotide
gcggccgctgaggcgcgcctactgagtcattagggactttcaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagcctcaggacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagctt

(19) United States
(12) Patent Application Publication
(10) Pub. No.: US 2009/0181424 A1
(43) Pub. Date: Jul. 16, 2009

(54) Mammalian Expression Vector Comprising the MCMV Promoter and First Intron of HCMV Major Immediate Early Gene

(76) Inventors: Fernando Albericio, Barcelona (ES); Luis Javier Cruz, Barcelona (ES); Fayna Garcia-Martin, Las Rozas C.P. Madrid (ES); Judit Tulla-Puche, Barcelona (ES)

Correspondence Address: FISHER, CHRISTEN & SABOL, 1120 20TH STREET, NW, SOUTH TOWER, SUITE 750, WASHINGTON, DC, 20036, US

(21) Appl. No.: 11/887,652

(22) PCT Filed: Apr. 20, 2006
(86) PCT No.: PCT/EP2006/003641

§ 371 Date: Sep. 28, 2007

(30) Foreign Application Priority Data
Apr. 22, 2005 (GB) 0508154.2
Apr. 26, 2005 (GB) 0508802.6

(51) Int. Cl. C12N 15/85; C12N 5/10; C12P 21/02

(52) U.S. Cl. 435/69.1; 435/320.1; 435/325; 435/358

(57) ABSTRACT

A mammalian expression vector that is a murine CMV promoter and the first intron of the major immediate early gene of the human cytomegalovirus. There are mammalian host cells containing the expression vector. There is also a process for the production of recombinant protein by using the expression vector.

The present invention relates to a mammalian expression vector comprising a murine CMV promoter and the first intron of the major immediate early gene of the human cytomegalovirus, CHO cells and CHO cell line containing this expression vector and a process for the production of a recombinant protein by using this expression vector.

The Chinese Hamster ovary cell (CHO) mammalian expression system is widely used in production of recombinant protein. Apart from lymphoid cell lines such as NS-0, it is one of the few cell types allowing for simple and efficient high-density suspension batch culture of animal cells. Furthermore, CHO cells allow for very high product yields and are comparatively robust to metabolic stresses whereas lymphoid cells are more difficult to culture at an industrial scale. In view of considerable costs of production, it is of utmost importance to maximize the yield of recombinant protein per bioreactor run. Choice of culture medium composition and bioreactor design and operation are parameters that impact yield and may be quite complex to optimize. Other factors which significantly affect the amount of polypeptide produced by a cell line are the gene copy number, the efficiency with which the gene is transcribed and the mRNA is translated, stability of the mRNA and the efficiency of secretion of the protein. Therefore, increases in the strength or transcriptional activity of the promoter controlling expression of product protein enhance yield. Incremental increases at the single cell level will translate into considerable improvements of product yield in high-density batch or fed-batch culture showing stationary phase gene expression at cell densities in the range of 10⁶ to 10⁷ cells/ml.

For transduction of mammalian cells, the majority of gene transfer experiments to date have used viral vectors encoding transgenes under the control of promoter elements derived from viruses. One of the most frequently used promoters in these expression cassettes is that of the human cytomegalovirus (hCMV) immediate early (IE) gene. The enhancer/promoter of this gene directs high levels of transgene expression in a wide variety of cell types. The activity of this promoter depends on a series of 17, 18, 19 and 21 bp imperfect repeats, some of which bind transcription factors of the NF-κB cAMP responsive binding protein (CREB) and the nuclear factor-1 families. A disadvantage, however, of the hCMV IE promoter is its pronounced species preference.

U.S. Pat. No. 5,866,359 describes a method of enhancing expression from an already strong hCMV promoter in CHO and NSO cells by co-expressing adenoviral E1A protein from a weak promoter. E1A is a multifunctional transcription factor which may act on cell cycle regulation and has both independent transcriptional activating and repressing functional domains. Fine tuning of E1A expression is crucial to achieve the ideal balance between gene transactivation and any negative impact on cell cycle progression. However, unwanted overexpression of E1A expression could reduce the capacity of the cell to synthesise the recombinant protein of interest.

U.S. Pat. No. 5,591,639 describes vectors containing the promoter, enhancer and complete 5′-untranslated region of the major immediate early gene of the human cytomegalovirus (hCMV-MIE) including intron A upstream of a heterologous gene. This approx. 2100 bp DNA sequence results in high level expression of several heterologous gene products. However, Chapman et al., Nucleic Acids Research, 19 (1991), 3979-3986, report that when the first 400 bp of this human sequence were present in expression plasmids, poor expression of glycoproteins was observed in both monkey kidney cells (COS7) and Chinese hamster ovary cells (DXB11). Deletion of these upstream modulatory sequences led to higher levels of expression for several mammalian glycoproteins in these cell types. Furthermore, a comparison of the SV40 early and hCMV immediate-early promoters/enhancers showed that the activity of the hCMV promoter can be increased by inserting intron A of the major immediate early gene of the human cytomegalovirus.

It is known that the transcriptional activity of the promoter of the major immediate early gene of the mouse cytomegalovirus (mCMV IE promoter) in CHO cells is much higher than that of the hCMV promoter. The mCMV IE promoter is able to drive high levels of expression without the pronounced species preference observed for the hCMV IE promoter (Addison, et al. Journal of General Virology (78 (1997), 1653-1661). However, attempts to enhance the activity of the mCMV promoter analogously to the hCMV promoter by inserting the natural first intron of the murine major immediate early gene downstream of the mCMV promoter failed. In contrast to the situation with hCMV promoter (cf U.S. Pat. No. 5,591,639), such natural first intron of mCMV was found to decrease significantly the expression of a recombinant gene from the mCMV promoter (cf. WO 2004/009823 A1)

In the art there is still a need to enhance the activity of the mCMV promoter. Therefore, the technical problem underlying the present invention is to provide a mCMV promoter based expression system which allows for enhanced protein expression from the mCMV promoter in mammalian host cells, in particular CHO cells.

According to the present invention, this technical problem is solved by providing a mammalian expression vector comprising a murine CMV promoter and the first intron of the major immediate early gene of the human cytomegalovirus (first hCMV intron) operably linked to a heterologous gene sequence encoding a desired recombinant protein. The mCMV promoter plus the first hCMV intron located downstream of the mCMV promoter form a regulatory unit which drives the expression of the downstream coding sequence. The mammalian expression vector of the present invention is a particular useful expression vector construct for high level expression of recombinant gene products in CHO cells. Surprisingly, the present vector comprising within the expression cassette the mCMV promoter in combination with the first hCMV intron drives the expression of heterologous protein products at levels higher than those seen for vectors only containing the mCMV promoter. The levels of heterologous proteins expressed by the mCMV promoter plus the first hCMV intron are at least equivalent to the protein levels expressed by the hCMV promoter plus the first hCMV intron. Not wishing to be bound by any particular theory, it is believed that the presence of the first hCMV intron obviously promotes efficient protein synthesis from the corresponding mRNA. These finding are unexpected and surprising in view of the fact that the activity of the mCMV promoter could not be increased by combining with the first mCMV intron.

In the context of the present invention a “mammalian expression vector” is a, preferably isolated and purified, DNA molecule which upon transfection into an appropriate mammalian host cell provides for a high level expression of a recombinant gene product within the host cell. In addition to the DNA sequence coding for the recombinant or heterologous gene product the expression vector comprises regulatory DNA sequences that are required for an efficient transcription of mRNAs from the coding sequence and an efficient translation of the mRNAs in the host cell line. In particular the expression vector according to the invention contains at least one regulatory unit comprising at least one mCMV promoter sequence in combination with the first intron (intron A) of the major immediate early gene of the human cytomegalovirus which is operably linked to the sequence coding for the recombinant protein and drives the expression of the encoded protein. The regulatory unit comprising the mCMV promoter plus the first hCMV intron is either directly linked to the coding sequence of the heterologous gene or is separated therefrom by intervening DNA such as for example by the 5′-untranslated region of the heterologous gene or a part therefrom.

According to the present invention the promoter of the mammalian expression vector is that of the major immediate early gene of the murine cytomegalovirus (mCMV IE or mCMV promoter). The murine CMV (mCMV) IE promoter was originally described by Dorsch-Hasler et al., Proc. Natl. Acad. Sci. USA, 82 (1985), 8325-8329, the entire disclosure of which is incorporated by reference into the present text.

Murine cytomegalovirus (mCMV) is a member of the highly diverse group of herpesviridae. Even amongst cytomegaloviruses of different host species there can be wide variation. For example, mCMV differs considerably from the human cytomegalovirus (hCMV) with respect to biological properties, immediate early (IE) gene organization, and overall nucleotide sequence. The 235-kbp genome of mCMV also lacks large internal and terminal repeat characteristics of hCMV. Accordingly, no isomeric forms of the mCMV genome exist (Ebeling, A. et al., (1983), J. Virol. 47, 421-433; Mercer, J. A. et al., (1983), Virology 129, 94-106).

A “promoter” is defined as a DNA sequence that mediates the initiation of transcription by directing RNA polymerase to bind to DNA and initiating RNA synthesis. It is known that the mCMV promoter is a strong promoter, i.e. a promoter which causes transcription to be initiated at high frequency. Furthermore it is known that the presence of the mCMV promoter in a vector will enhance transfection rates in CHO cells, preferably when using an expression vector comprising a first transcription unit for an heterologous gene which first unit is giving rise to the protein product upon expression in a host cell and which first transcription unit is under the control of the mCMV promoter, and further comprising a second transcription unit comprising a glutamine synthetase (GS) marker gene.

According to the invention the promoter employed can also be a functional fragment or a functional sequence variant of the mCMV promoter. Thus, any sequence variant or fragment of the mCMV promoter that is functional in or capable of mediating the initiation of transcription and thus can drive transiently or stably the expression of the recombinant or heterologous product gene can be used as mCMV promoter. “Functional variants” of the mCMV promoter include inter alia base insertions, deletions or point mutations of the natural mCMV sequence and can be generated by methods well-known in the art, e.g. by primer-directed PCR, ‘error-prone’ PCR, ‘gene-shuffling’ termed PCR-reassembly of overlapping DNA fragments or by in-vivo random mutagenesis of bacterial clones followed by library transfection and functional selection in CHO cells. For instance, random mutagenesis can be achieved by alkylating chemicals or UV-irradiations as described in Miller, J., Experiments in Molecular Genetics, Cold Spring Harbor Laboratory (1972). Optionally, a natural mutator-strain of a host bacterium may be used. Preferably, such variant sequence is at least 65%, more preferably 75%, most preferably 90% homologous in DNA sequence to the corresponding part of the natural murine CMV promoter. An example of a functional sequence variant of the mCMV promoter is a promoter sequence with a transcription start site which is engineered in order to create a suitable restriction site for insertion of the recombinant product gene.

In a preferred embodiment of the invention the mCMV promoter used essentially corresponds to a large approx. 2.1 kb PstI fragment described in U.S. Pat. No. 4,968,615. In another preferred embodiment, the mCMV promoter employed is a fragment thereof which comprises the transcription start site (+0) and extends upstream to about position −500. In another preferred embodiment, a core promoter region is employed that extends from the transcription start site upstream but to the Xho I restriction site at about position −150 from the natural transcription start site or even extending but to position −100 upstream from the natural transcriptions start site. In still another preferred embodiment the mCMV promoter used is a fragment of the mCMV promoter from −491 to +36 or a fragment from −1336 to +36 as described by Addison et al., J. Gen. Virol., 78 (1997), 1653-1661.

In a preferred embodiment of the present invention the first hCMV intron (hCMV intron A or human CMV intron A) used essentially corresponds to the 823 bp sequence as defined in Chapman et al., Nucleic Acids Research, 19 (1991), 3979-3986.

According to the invention the first hCMV intron employed can also be a functional fragment or a functional sequence variant thereof. Thus, any sequence variant or fragment of the hCMV intron A that is functional in or capable of enhancing the transcriptional activity of the mCMV promoter can be used as hCMV intron A. “Functional variants” of the first hCMV intron include inter alia base insertions, deletions or point mutations of the natural mCMV sequence. A functional variant can have a sequence with single base modifications that make the translation initiation signal closer to the Kozak consensus sequence for translation initiation. Functional variants also include truncations of the 823 bp sequence as defined in Chapman et al., Nucleic Acids Research, 19 (1991), 3979-3986, whereby according to the invention the sequence of the functional variants has a length of at least 100-150 bp, preferably of at least 200-300 bp, more preferred of at least 400-500 bp and most preferred of at least 600-700 bp. According to the present invention the sequence of the functional variants shows over its entire length a homology of at least 60%, preferably of at least 70%, more preferred of at least 80% and most preferred of at least 90% or 95% to the 823 bp sequence as defined in Chapman et al., Nucleic Acids Research, 19 (1991), 3979-3986.

In the context of the invention the terms “heterologous coding sequence”, “heterologous gene sequence”, “heterologous gene”, “recombinant gene”, “gene of interest” and “transgene” are used interchangeably. By these terms a DNA sequence means one that codes for a recombinant or heterologous gene product. The heterologous gene sequence is naturally not present in the host cell and is derived from an organism of a different species. A recombinant or heterologous gene product according to the present invention is the recombinant protein that is sought to be expressed in the mammalian cell and harvested in high amount. The gene product may also be a peptide or polypeptide. It may be any protein of interest, e.g. a therapeutic protein such as an interleukin or an enzyme or a subunit of a multimeric protein such as an antibody or a fragment thereof. The recombinant product gene may include a signal sequence that encodes a signal peptide allowing secretion of the once expressed polypeptide from the host producer cell. Thus, in a further preferred embodiment of the present invention, the product protein is a secreted protein. More preferably, the product protein is an antibody or engineered antibody or a fragment thereof, most preferably it is an Immunoglobulin G (IgG) antibody.

In another preferred embodiment of the invention the mammalian expression vector further comprises a portion from the murine IgG2A locus DNA which portion is further enhancing the activity of the mCMV promoter as disclosed in WO 2004/009823 A1 which is hereby incorporated by reference. From WO 2004/009823 A1 it is known that the murine IgG 2A targeting sequence even improved gene expression in CHO cells upon transient transfection of CHO cells with expression vectors. Preferably the portion from the murine IgG2A locus DNA is a 5.1 kb BamHI genomic fragment which includes all of the coding region of murine Ig gamma 2A except the most 5′ part of the CHI exon (Yamawaki-Kataoka, Y. et al., Proc. Natl. Acad. Sci. U.S.A. (1982) 79: 2623-2627; Hall, B. et al., Molecular Immunology (1989) 26:819-826; Yamawaki-Kataoka, Y. et al., Nucleic Acid Research (1981) 9: 1365-1381).

Preferably, the expression vectors of the invention also contain a limited number of useful restriction sites for insertion of the expression cassette comprising the recombinant gene under control of the mCMV promoter plus first hCMV intron sequence. Where used in particular for transient/episomal expression only, the expression vectors of the invention may further comprise an origin of replication such as origin of Epstein Barr Virus (EBV) or SV40 virus for autonomous replication/episomal maintenance in eukaryotic host cells but may be devoid of a selectable marker. Expression vectors of the invention can be for example, without being limited to, linear DNA fragments, DNA fragments encompassing nuclear targeting sequences or may be specially optimized for interaction with transfection reagents, animal viruses or suitable plasmids that can be shuttled and produced in bacteria.

Preferably, the mammalian expression vectors of the present invention further contain at least one expressible marker selectable in animal cells. Any selection marker commonly employed such as thymidine kinase (tk), dihydrofolate reductase (DHFR) or glutamine synthetase (GS) may be used. In a preferred embodiment, an expressible GS selection marker is employed (Bebbington et al., 1992, High-level expression of a recombinant antibody from myeloma cells using a glutamine synthetase gene as an amplifiable selectable marker, Bio/Technology 10:169-175; Cockett et al., 1990, High level expression of tissue inhibitor of metalloproteinases in Chinese Hamster Ovary (CHO) cells using Glutamine synthetase gene amplification, Bio/Technology 8: 662-667). The GS-system is one of only two systems that are of particular importance for the production of therapeutic proteins. In comparison to the dihydrofolate reductase (DHFR) system, the GS system offers a large time advantage during development because highly productive cell lines can often be created from the initial tranfectant thus avoiding the need for multiple rounds of selection in the presence of increasing concentrations of selective agent in order to achieve gene amplification (Brown et al., 1992, Process development for the production of recombinant antibodies using the glutamine synthetase (GS) system, Cytotechology 9:231-236). It goes without saying that equivalent to a second transcription unit for expression of the marker gene, an expression unit could use a monocistronic expression cassette both for the product gene and the marker gene by employing e.g. internal ribosome entry sites as is routinely employed in the art.

In a preferred embodiment of the inventive mammalian expression vector the recombinant or heterologous gene product and the selectable marker are produced from a single dicistronic transcription unit. That is, the regulatory unit consisting of the mCMV promoter and the first hCMV intron drives both the expression of the downstream located heterologous or recombinant gene sequence and the expression of the also downstream located selectable marker. It is known that dicistronic vectors efficiently coamplify both the selectable marker and the recombinant gene. Also, expression of two open reading frames from a single transcription unit has been shown to yield high levels of protein production. In another preferred embodiment of the inventive mammalian expression vector the gene of interest, i.e. the recombinant or heterologous gene, and the selectable marker gene are on separate transcription units, i.e. their expression is driven by separate promoters. In this embodiment the selectable marker gene can also be under the control of the regulatory unit consisting of the mCMV promoter and the first hCMV intron. However, the expression of the selectable marker can also be driven by another promoter, e.g. one of the SV40 early and late promoters, the hybrid Moloney murine leukaemia virus-SV40 promoter SRM or the hCMV promoter.

Another preferred embodiment of the present invention relates to a mammalian expression vector which comprises at least two separate transcription units. Such an expression vector is also referred as double-gene vector. In a preferred embodiment each of the first and second transcription units comprises a different recombinant gene of interest. Preferably, the first transcription unit comprises a first product gene or heterologous coding sequence under the control of the regulatory unit of the invention, i.e. the mCMV promoter plus the first hCMV intron, giving rise to a first product protein upon expression in a host cell, and the second transcription unit comprises a second product gene or heterologous coding sequence under the control of the regulatory unit of the invention, giving rise to a second product protein upon expression in a host cell. An example for such a vector is a double gene vector, in which the first transcription unit comprises a gene encoding the heavy chain of an antibody and the second transcription unit comprises a gene encoding the light chain of this antibody. However, it is also possible that the expression of the second transcription unit comprising a recombinant gene sequence is driven by a regulatory unit other than the inventive regulatory unit. The second transcription unit may for example include the hCMV promoter.

In another preferred embodiment of the invention the mammalian expression vector comprises at least a (first) transcription unit for a product gene, giving rise to product protein upon expression in a host cell, and which transcription unit is under the control of the regulatory unit of the invention, i.e. the mCMV promoter plus the first hCMV intron, and further comprising a second transcription unit comprising a marker gene, preferably a glutamine synthetase (GS) marker gene. The product gene or gene of interest (GOI) can be e.g. an immunoglobulin coding sequence. A glutamine synthetase marker gene is any enzymatically active GS coding sequence, be it a natural gene sequence or a variant thereof. The above definitions of “functional variant” as set forth above apply here as well including the preferred ranges of sequence homology. Such an expression vector allows for much higher transfection rates upon transfection in CHO cells than does e.g. an expression vector in which the first transcription unit harbouring the gene of interest is under control of the hCMV promoter. This despite the fact that in CHO cells, transcriptional activity of the mCMV promoter is much higher than that of hCMV promoter; usually it is believed that upon transfection, higher metabolic load reduces clonal survival upon transfection, resulting in lower numbers of transfectants. Thus the effect can not be correlated in an obvious manner with the amount or unexpected toxicity of product protein expressed the latter possibly adversely affecting growth of transfectants.

Another aspect of the present invention relates to a regulatory unit comprising the mCMV promoter plus the first hCMV intron located downstream of the mCMV promoter sequence. When the regulatory unit of the invention is operably linked to a gene sequence it will mediate the initiation of transcription of this gene sequence and will stabilise RNA transcripts and promote efficient protein synthesis from the corresponding mRNA within the environment of a mammalian cell. Preferably the regulatory unit of the invention is flanked by one or more suitable restriction sites in order to enable the insertion of the regulatory unit into a vector upstream of a sequence coding for a heterologous gene product and/or its release from a vector. In a preferred embodiment of the invention the regulatory unit is flanked upstream by an AscI restriction site and downstream by a Hind III restriction site. Thus, the regulatory unit according to the present invention can be used for the construction of an expression vector, in particular a mammalian expression vector.

Still another aspect of the present invention relates to an expression cassette comprising the regulatory unit of the invention and a transcription unit, i.e. a DNA sequence encoding a recombinant or heterologous protein product. The regulatory unit is located upstream of the transcription unit and is operably linked thereto. The regulatory unit of the invention is either directly linked to the transcription unit, i.e. coding sequence of the heterologous gene or is separated therefrom by intervening DNA such as for example by the 5′-untranslated region of the heterologous gene. Preferably the expression cassette is flanked by one or more suitable restriction sites in order to enable the insertion of the expression cassette into a vector and/or its excision from a vector. Thus, the expression cassette according to the present invention can be used for the construction of an expression vector, in particular a mammalian expression vector.

A further aspect of the present invention relates to a mammalian host cell containing the mammalian expression vector according to the invention. The mammalian host cell can be a human or non-human cell. Preferred examples of the mammalian host cells include, without being restricted to, MRC5 human fibroblasts, 983M human melanoma cells, MDCK canine kidney cells, RF cultured rat lung fibroblasts isolated from Sprague-Dawley rats, B16BL6 murine melanoma cells, P815 murine mastocytoma cells and MTIA2 murine mammary adenocarcinoma cells. In a particular preferred embodiment the mammalian host cell is a Chinese hamster ovary (CHO) cell or cell line (Puck et al., 1958, J. Exp. Med. 108: 945-955). Suitable CHO cell lines include e.g. CHO K1 (ATCC CCL-61), CHO pro3-, CHO DG44, CHO P12 or the dhfr-CHO cell line DUK-BII (Chassin et al., PNAS 77, 1980, 4216-4220) or DUXB11 (Simonsen et al., PNAS 80, 1983, 2495-2499).

For introducing the expression vector into an mammalian host cell according to the present invention any transfection technique such as those well-known in the art, e.g. electoporation, calcium phosphate co-precipitation, DEAE-dextran transfection, lipofection, can be employed if appropriate for a given host cell type. It is to be noted that the mammalian host cell transfected with the vector of the present invention is to be construed as being a transiently or stably transfected cell line. Thus, according to the present invention the present mammalian expression vector can be maintained episomally or can be stably integrated in the genome of the mammalian host cell.

A transient transfection is characterised by non-appliance of any selection pressure for a vector borne selection marker. A pool or batch of cells originating from a transient transfection is a pooled cell population that comprises cells which have taken up and do express and cells that have not taken up the foreign DNA. In transient expression experiments which commonly last 20-50 hours post transfection, the transfected vectors are maintained as episomal elements and are not yet integrated into the genome. That is the transfected DNA, does not usually integrate into the host cell genome. The host cells tend to lose the transfected DNA and overgrow transfected cells in the population upon culture of the transiently transfected cell pool. Therefore expression is strongest in the period immediately following transfection and decreases with time. Preferably, a transient transfectant according to the present invention is understood as a cell that is maintained in cell culture in the absence of selection pressure up to a time of 90 hours post transfection.

In a preferred embodiment of the invention the mammalian host cell e.g. the CHO host cell is stably transfected with the mammalian expression vector of the invention. Stable transfection means that newly introduced foreign DNA such as vector DNA is becoming incorporated into genomic DNA, usually by random, non-homologous recombination events. The copy number of the vector DNA and concomitantly the amount of the gene product can be increased by selecting for cell lines in which the vector sequences have been amplified after integration into the DNA of the host cell. Therefore, it is possible that such stable integration gives rise, upon exposure to further selection pressure for gene amplification, to double minute chromosomes in CHO cells. Furthermore, in case of a vector sequence, a stable transfection may result in loss of vector sequence parts not directly related to expression of the recombinant gene product, such as e.g. bacterial copy number control regions rendered superfluous upon genomic integration. Therefore, a transfected host cell has integrated at least part or different parts of the expression vector into the genome. Likewise, transfection of CHO cells with two or several DNA fragments giving rise at least in vivo to functional equivalents of the essential elements of the mammalian expression vector of the invention, namely the heterologous gene product under control of the murine CMV promoter and the first hCMV intron, is contained in the definition of such transfected host cells. In vivo assembly of functional DNA sequences after transfection of fragmented DNA is described e.g. in WO 99/53046.

A further aspect of the present invention relates to a process for the production of a recombinant protein, comprising the steps of

a) transfecting a mammalian host cell or host cell line with an expression vector comprising a murine CMV promoter and the first hCMV intron operably linked to a sequence coding for a recombinant protein
b) culturing the cell under appropriate conditions to enable growth and/or propagation of the cell and expression/production of the recombinant protein and
c) harvesting the recombinant protein produced.

Methods for transfecting a mammalian host cell or mammalian host cell line with a vector are well known in the art. In the process according to the invention any transfection technique such as those well-known in the art, e.g. electroporation, calcium-phosphate co-precipitation, DEAE-dextran transfection, lipofection, can be employed if appropriate for a given host cell type. According to the invention the host cell can be stably or transiently transfected with the expression vector.

The terms “host cell” or “host cell line” refer to any cells, in particular mammalian cells, which are capable of growing in culture and expressing a desired protein recombinant product protein. The mammalian host cell used in the process of the invention can be a human or non-human cell. In a preferred embodiment the mammalian host cell line used for transfection can be any Chinese hamster ovary (CHO) cell line (Puck et al., 1958, J. Exp. Med. 108: 945-955). Suitable cell lines include e.g. CHO K1 (ATCC CCL-61), CHO pro3-, CHO DG44, CHO P12 or the dhfr-CHO cell line DUK-BII (Chassin et al., PNAS 77, 1980, 4216-4220) or DUXB11 (Simonsen et al., PNAS 80, 1983, 2495-2499). It is known that the transcriptional activity of the promoter of the major immediate early gene of the mouse cytomegalovirus (mCMV promoter) is very high in CHO cells. Furthermore, if the mammalian expression vector of the present invention comprises the murine IgG2a sequence this sequence will even improve gene expression in CHO cells upon transient transfection of CHO cells with the expression vector according to the present invention.

Suitable media and culture methods for mammalian cell lines are well-known in the art, as described in U.S. Pat. No. 5,633,162 for instance. Examples of standard cell culture media for laboratory flask or low density cell culture and being adapted to the needs of particular cell types include, without being restricted to, Roswell Park Memorial Institute (RPMI) 1640 medium (Morre, G., The Journal of the American Medical Association, 199, p. 519 f. 1967), L-15 medium (Leibovitz, A. et al., Amer. J. of Hygiene, 78, 1 p. 173 ff, 1963), Dulbecco's modified Eagle's medium (DMEM), Eagle's minimal essential medium (MEM), Ham's F12 medium (Ham, R. et al., Proc. Natl. Acad. Sc. 53, p 288 ff. 1965) or Iscoves' modified DMEM lacking albumin, transferrin and lecithin (Iscoves et al., J. Exp. med. 1, p. 923 ff., 1978). For instance, Ham's F10 or F12 media were specially designed for CHO cell culture. Other media specially adapted to CHO cell culture are described in EP-481 791. It is known that such culture media can be supplemented with fetal bovine serum (FBS, also called fetal calf serum FCS), the latter providing a natural source of a plethora of hormones and growth factors. The cell culture of mammalian cells is nowadays a routine operation well-described in scientific textbooks and manuals, it is covered in detail e.g. in R. Ian Fresney, Culture of Animal cells, a manual, 4^{th} edition, Wiley-Liss/N.Y., 2000.

In a preferred embodiment of the present invention the cell culture medium used is devoid of fetal calf serum (FCS or FBS), which then is being termed ‘serum-free’. Cells in serum-free medium generally require insulin and transferrin in a serum-free medium for optimal growth. Transferrin may at least partially be substituted by non-peptide chelating agents or siderophores such as tropolone as described in WO 94/02592 or increased levels of a source of an organic iron favorably in conjunction with antioxidants such as vitamin C. Most cell lines require one or more of synthetic growth factors (comprising recombinant polypeptides), including e.g. epidermal growth factor (EGF), fibroblast growth factor (FGF), insulin like growth factors I and II (IGFI, IGFII), etc. Other classes of factors which may be necessary include: prostaglandins, transport and binding proteins (e.g. ceruloplasmin, high and low density lipoproteins, bovine serum albumin (BSA)), hormones, including steroid-hormones, and fatty acids. Polypeptide factor testing is best done in a stepwise fashion testing new polypeptide factors in the presence of those found to be growth stimulatory. Those growth factors are synthetic or recombinant. There a several methodological approaches well-known in animal cell culture, an exemplary being described in the following. The initial step is to obtain conditions where the cells will survive and/or grow slowly for 3-6 days after transfer from serum-supplemented culture medium. In most cell types, this is at least in part a function of inoculum density. Once the optimal hormone/growth factor/polypeptide supplement is found, the inoculum density required for survival will decrease.

In another preferred embodiment, the cell culture medium is protein-free, that is free both of fetal serum and individual protein growth factor supplements or other protein such as recombinant transferrin.

In another embodiment the process of the present invention directed to expression and harvest of the recombinant product protein includes a high-density growth of the animal host cells e.g. in an industrial fed-batch bioreactor. Conventional downstream processing may then be applied. Consequently, a high-density growth culture medium has to be employed. Such high-density growth media can usually be supplemented with nutrients such as all amino acids, energy sources such as glucose in the range given above, inorganic salts, vitamins, trace elements (defined as inorganic compounds usually present at final concentrations in the micromolar range), buffers, the four nucleosides or their corresponding nucleotides, antioxidants such as glutathione (reduced), vitamin C and other components such as important membrane lipids, e.g. cholesterol or phosphatidylcholine or lipid precursors, e.g. choline or inositol. A high-density medium will be enriched in most or all of these compounds, and will, except for the inorganic salts based on which the osmolarity of the essentially isotonic medium is regulated, comprise them in higher amounts (fortified) than the afore mentioned standard media as can be incurred from GB2251 249 in comparison with RPMI 1640. Preferably, a high-density culture medium according to the present invention is fortified in that all amino acids except for tryptophan are in excess of 75 mg/l culture medium. Preferably, in conjunction with the general amino acid requirement, glutamine and/or asparagine are in excess of 1 g/l, more preferably of 2 g/l of high-density culture medium. In the context of the present invention, high-density cell culture is defined as a population of animal cells having temporarily a density of viable cells of at least or in excess of 10⁵ cells/ml, preferably of at least or in excess of 10⁶ cells/ml, and which population has been continuously grown from a single cell or inoculum of lower viable cell density in a cell culture medium in a constant or increasing culture volume.

In a further preferred embodiment the process of the present invention includes a fed-batch culture. A fed-batch culture is a culture system wherein at least glutamine, optionally with one or several other amino acids, preferably glycine, is fed to the cell culture as described in GB2251249 for maintaining their concentration in the medium, apart from controlling glucose concentration by separate feed. More preferably, the feed of glutamine and optionally one or several other amino acids is combined with feeding one or more energy sources such as glucose to the cell culture as described in EP-229 809-A. Feed is usually initiated at 25-60 hours after start of the culture; for instance, it is useful to start feed when cells have reached a density of about 10⁶ cells/ml. It is well known in the art that in cultured animal cells, ‘glutaminolysis’ (McKeehan et al., 1984, Glutaminolysis in animal cells, in: Carbohydrate Metabolism in Cultured Cells, ed. M. J. Morgan, Plenum Press, New York, pp. 11-150) may become an important source of energy during growth phase. The total glutamine and/or asparagine feed (for substitution of glutamine by asparagine, see Kurano, N. et al., 1990, J. Biotechnology 15, 113-128) is usually in the range from 0.5 to 10 g per 1, preferably from 1 to 2 g per I culture volume; other amino acids that can be present in the feed are from 10 to 300 mg total feed per litre of culture, in particular glycine, lysine, arginine, valine, isoleucine and leucine are usually fed at higher amounts of at least 150 to 200 mg as compared to the other amino acids. The feed can be added as shot-addition or as continuously pumped feed, preferably the feed is almost continuously pumped into the bioreactor. It goes without saying that the pH is carefully controlled during fed-batch cultivation in a bioreactor at an approximately physiological pH optimal for a given cell line by addition of base or buffer. When glucose is used as an energy source the total glucose feed is usually from 1 to 10, preferably from 3 to 6 grams per litre of the culture. Apart from inclusion of amino acids, the feed preferably comprises a low amount of choline in the range of 5 to 20 mg per litre of culture. More preferably, such feed of choline is combined with supplementation of ethanolamine essentially as described in U.S. Pat. No. 6,048,728, in particular in combination with feeding glutamine. It goes without saying that upon use of the GS-marker system, lower amounts of glutamine will be required as compared to a non-GS expression system since accumulation of excessive glutamine in addition to the endogenously produced would give rise to ammonia production and concomitant toxicity. For GS, glutamine in the medium or feed is mostly substituted by its equivalents and/or precursors, that is asparagine and/or glutamate.

Methods for harvesting, i.e. isolating and/or purifying a given protein from a cell, a cell culture or the medium in which the cells had been cultured are well known in the art. Proteins can be isolated and/or purified from biological material for example by fractionated precipitation with salts or organic solvents, ion exchange chromatography, gel chromatography, HPLC, affinity chromatography etc.

Still another aspect of the present invention relates to a process for increasing the activity of a mCMV promoter for expressing a recombinant or heterologous gene product in a mammalian host cell by using the first hCMV intron. According to this process of the present invention the expression of a desired heterologous gene product from the mCMV promoter can be markedly enhanced by inserting the hCMV intron A sequence by molecular cloning methods into an already existing vector molecule between the mCMV promoter sequence and the gene sequence coding for the desired protein such that the mCMV promoter and first hCMV intron are operably linked to the heterologous gene sequence. Molecular cloning methods are well-known in the art and are for example described in Maniatis, T., Fritsch, E. F. and Sambrook, J., Molecular cloning: A laboratory manual, Cold Spring Harbor Laboratory Press, Cold Spring Harbor, N.Y. (1989). Afterwards a mammalian host cell such as a CHO cell is transfected with the thus created vector construct comprising the first hCMV intron between the mCMV promoter and the downstream located heterologous DNA sequence encoding the desired protein. Transfectants are then cultured under appropriate conditions in order to enable growth and/or propagation of the cells and expression/production of the recombinant protein. Finally the recombinant protein produced is harvested, i.e. isolated and purified. By the use of the first hCMV intron in the process of the present invention it is possible to enhance significantly the efficiency with which a recombinant-protein is expressed from a mCMV promoter and to obtain a higher level expression of the recombinant protein. According to the present invention it is thus possible to improve the expression efficiency of already existing expression vectors in which the expression of the recombinant gene is driven only by the mCMV promoter.

The present invention, therefore, also relates to the use of the first hCMV intron for improving the activity of mCMV promoter.

Preferred embodiments of the invention are illustrated in the figures. What is shown is:

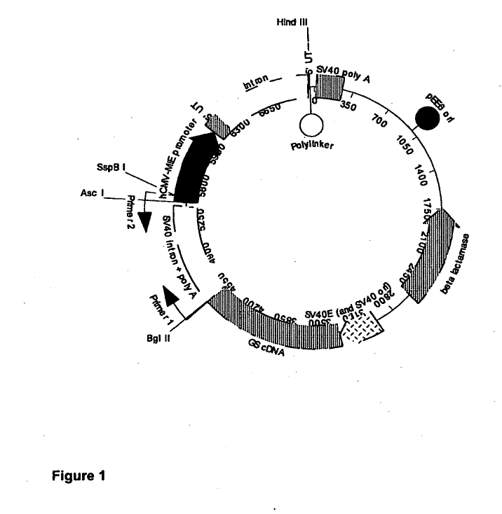

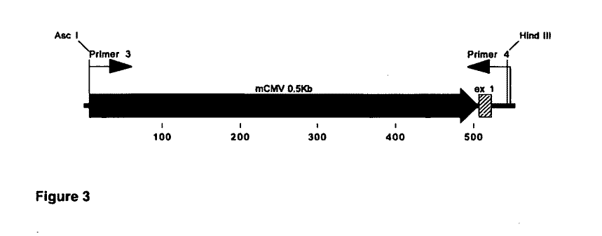

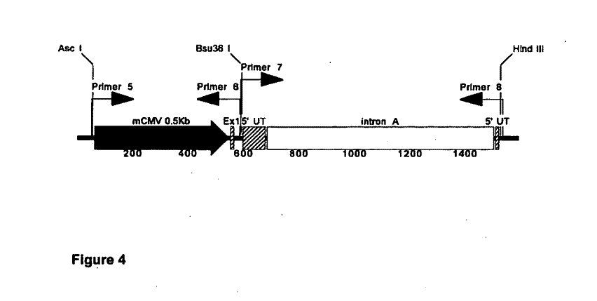

FIG. 1 Physical map of vector Delta-pEE12.4 used for cloning the mCMV (short)-Ex1 PCR product comprising the short mCMV promoter as depicted in FIG. 3 and mCMV-human Intron A fragment comprising the short mCMV promoter and human intron A as depicted in FIG. 4.

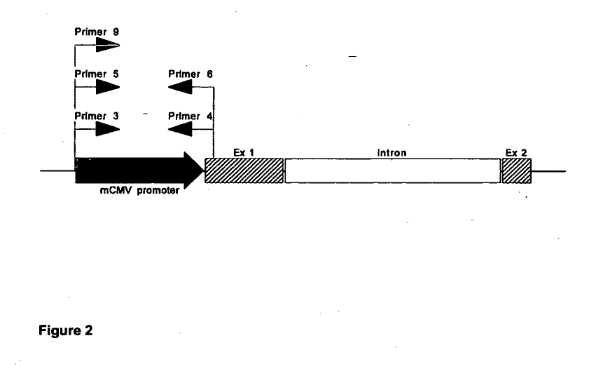

FIG. 2 Physical map of the mCMV template as used for PCR amplification of the mCMV promoter wherein position of various primers used are indicated.

FIG. 3 Physical map of the mCMV (short)-Ex 1 PCR product comprising the short mCMV promoter used for joining with the Intron A-PCR fragment in order to obtain fragment mCMV-human Intron A, comprising the mCMV promoter+the first human CMV intron.

FIG. 4 Physical map of fragment mCMV-human Intron A comprising the mCMV promoter+the first human CMV intron.

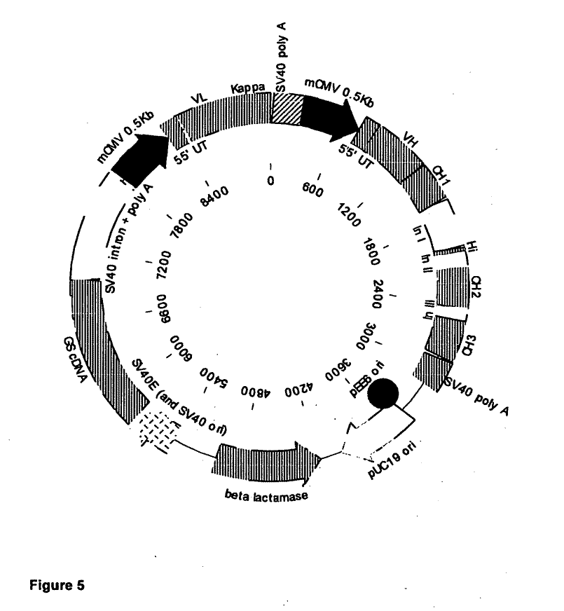

FIG. 5 Plasmid map of the double-gene expression vector pcB72.3-mCMV for expressing a human IgG4/Kappa antibody wherein the expression of the antibody is under the control of the short mCMV promoter. The vector further comprises the selectable GS marker gene.

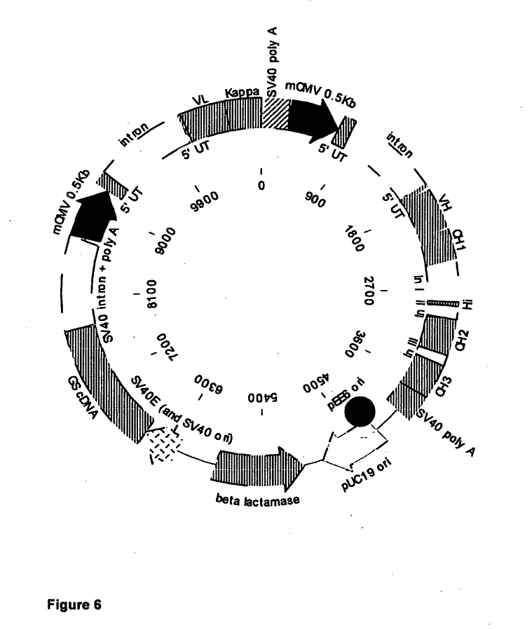

FIG. 6 Plasmid map of the double-gene expression vector pcB72.3-mCMV+intron A for expressing a human IgG4/Kappa antibody wherein the expression of the antibody is under the control of the short mCMV promoter plus the first hCMV intron. The vector further comprises the selectable GS marker gene.

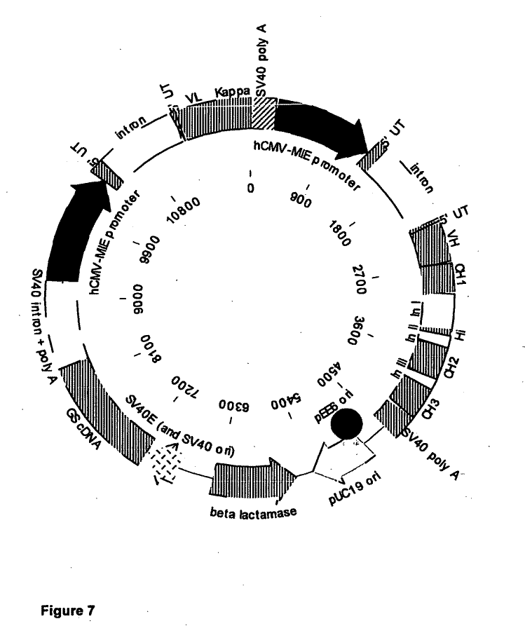

FIG. 7 Plasmid map of the double-gene expression vector pcB72.3 for expressing a human IgG4/Kappa antibody wherein the expression of the antibody is under the control of the hCMV promoter plus the first hCMV intron (control). The vector further comprises the selectable GS marker gene.

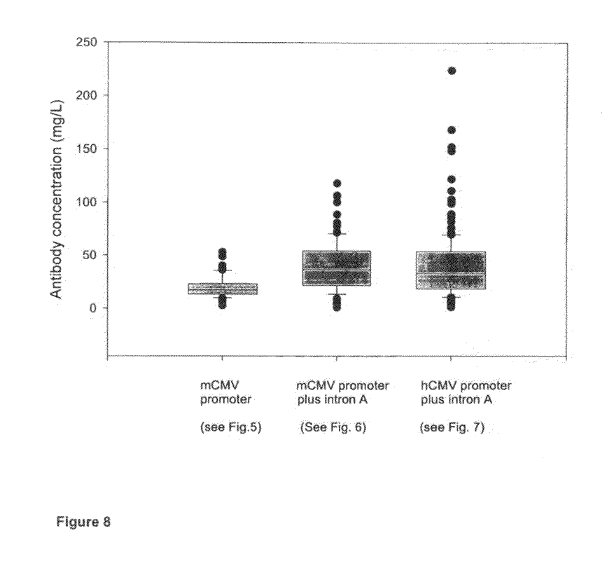

FIG. 8 Relative expression levels of the IgG4/Kappa antibody in stably transfected CHO-K1SV cells the short mCMV promoter only, the mCMV promoter plus first hCMV intron, and from the hCMV promoter plus the first hCMV intron (control).

It is understood that the explanations and references made to a given preferred embodiment in the present specification of the invention relate likewise to all further preferred embodiment of the present invention.

The present invention is explained in more detail by the following examples.

### EXAMPLES

### Materials and Methods

### Cells Used

CHO cell line CHOK1SV: is a variant of the cell line CHO-K1 and has been adapted to growth in suspension and protein-free medium.

Propagation of CHOK1SV cells:

CHOK1SV cells were routinely propagated in suspension shaker flasks in CD-CHO medium (Invitrogen) supplemented with 6 mM L-glutamine. Seed concentration was 2×10⁵ cells/ml, and cells are split every 4 days. Flasks were gassed with 5% CO₂ and incubated at 36.5° C. (between 35.5° C. and 37.0° C.) with orbital shaking at 140 rpm.

### Stable Transfections:

Cells used for transfections were grown in cell suspension culture, as detailed before. Cells from growing cultures were centrifuged and washed once in serum-free medium prior to being re-suspended to a concentration of 1.43×10⁷ cells/mL. A 0.7 mL volume of the cell suspension and 40 μg of plasmid DNA were added to an electroporation cuvette. The cuvette was then placed in the electroporation apparatus and a single pulse of 250 V and 400 μF was delivered. Following transfection, the cells were distributed into 96-well plates at approximately 2,500 host cells/well (5×10⁴/mL), using the non-selective DMEM-based medium supplemented with 10% dFCS. The plates were incubated at 36.5° C. (between 35.5° C. and 37.0° C.) in an atmosphere of 10% CO₂ in air.

The day after the transfection, DMEM-based medium supplemented with 10% dFCS/66 μM L-methionine sulphoximine was added to each well (150 μL/well) to give a final L-methionine sulphoximine concentration of 50 μM. The plates were monitored to determine when the non-transfected cells died and when foci of transfected cells appeared. Foci of transfected cells became apparent approximately three to four weeks after transfection. All the cell lines examined and progressed further came from wells containing only a single colony.

### Assessment of Productivity of Cell Lines in Static Culture

The 96-well transfection plates were incubated for approximately three weeks to allow colony formation. The resulting colonies were examined microscopically to verify that the colonies were of a suitable size for assay (covering greater than 60% of the bottom of the well), and that only one colony was present in each well.

Suitable colonies were transferred to wells of 24-well plates containing 1 mL of selective growth medium (DMEM-based medium/10% dFCS/25 μM L-methionine sulphoximine). These cultures were incubated for 14 days at 36.5° C. (between 35.5° C. and 37.0° C.) in an atmosphere of 10% CO₂ in air. The supernatant of each well was harvested and analysed for the concentration of antibody present by the protein-A HLPC method.

### Assembly ELISA:

The antibody concentration of samples was determined using a sandwich ELISA which measures assembled human IgG. This involved capture of samples and standard onto a 96 well plate coated with an anti-human Fc antibody. Bound antibody was revealed with an anti-human light chain linked to horseradish peroxidase and the chromogenic substrate TMB. Colour development was proportional to the concentration of antibody present in the sample when compared to the standard.

### Protein A HPLC:

The Protein A affinity chromatography method for the measurement of IgG was performed on an Agilent 100 HPLC. IgG product binds selectively to a Poros Protein A immunodetection column. Non-bound material is washed from the column and the remaining bound antibody is released by decreasing the pH of the solvent. The elution was monitored by absorbance at 280 nm and product was quantified (using Chemstation software) against a generic antibody standard and a correction is made for differences in extinction coefficients.

### Vector Construction

TABLE 1
table of vectors used in cloning procedures

| | |
|---|---|
| | Primer sequence (5′3′) |
| Primer | (Relevant restriction enzyme sites are |
| number | indicated in bold) |

| | |
|---|---|
| 1 | CCAGAGAGATCTTTGTGAAGG |
| 2 | GCGCGCTGTACATATTATGATATGGATACAACGTATGCAATGG |
| | CCAATAGCCAATATTGGCGCGCCTCACCGTCCTTGACACGAAG |
| | C |
| 3 | CGTATAGGCGCGCCTACTGAGTCATTAGGGACTTTCC |
| 4 | GCATCGAAGCTTCTGCGTTCTACGGTGGTCAGACC |
| 5 | GATCGGCGCGCCTAAGCTACTGAGTCATTAGGGACTTTC |
| 6 | GATCCCTGAGGCTGCGTTCTACGGTGGTC |
| 7 | GATCCCTCAGGACCTCCATAGAAGACACC |
| 8 | GATCAAGCTTCGTGTCAAGGACG |
| 9 | GATCGCTAGCGGCCGCTGAGGCGCGCCTACTGAG |

The sequences of primers 1 to 9 are shown as SEQ ID No. 1 to 9 in the sequence listing.

### Generation of Vector DELTA-PEE 12.4

Vector Delta-pEE12.4 was generated by replacing part of the hCMV promoter region between the RE sites Bgl II and SspB I within the GS vector pEE12.4 with a PCR fragment, that introduces an Asc I site at the 3′-end. For this, the forward primer 1 and the reverse primer 2 were used (see Table 1)

Both PCR primers were used in a PCR reaction with DNA of vector pEE12.4 as the DNA template. The PCR product has the following sequence (SEQ ID No. 10):

CCAGAGAGATCTTTGTGAAGGAACCTTACTTCTGTGGTGTGACATAATTG
GACAAACTACCTACAGAGATTTAAAGCTCTAAGGTAAATATAAAATTTTT
AAGTGTATAATGTGTTAAACTACTGATTCTAATTGTTTGTGTATTTTAGA
TTCCAACCTATGGAACTGATGAATGGGAGCAGTGGTGGAATGCCTTTAAT
GAGGAAAACCTGTTTTGCTCAGAAGAAATGCCATCTAGTGATGATGAGGC
TACTGCTGACTCTCAACATTCTACTCCTCCAAAAAAGAAGAGAAAGGTAG
AAGACCCCAAGGACTTTCCTTCAGAATTGCTAAGTTTTTTGAGTCATGCT
GTGTTTAGTAATAGAACTCTTGCTTGCTTTGCTATTTACACCACAAAGGA
AAAAGCTGCACTGCTATACAAGAAAATTATGGAAAAATATTCTGTAACCT
TTATAAGTAGGCATAACAGTTATAATCATAACATACTGTTTTTTCTTACT
CCACACAGGCATAGAGTGTCTGCTATTAATAACTATGCTCAAAAATTGTG
TACCTTTAGCTTTTTAATTTGTAAAGGGGTTAATAAGGAATATTTGATGT
ATAGTGCCTTGACTAGAGATCATAATCAGCCATACCACATTTGTAGAGGT
TTTACTTGCTTTAAAAAACCTCCCACACCTCCCCCTGAACCTGAAACATA
AAATGAATGCAATTGTTGTTGTTAACTTCTTTATTGCAGCTTATAATGGT
TACAAATAAAGCAATAGCATCACAAATTTCACAAATAAAGCATTTTTTTC
ACTGCATTCTAGTTGTGGTTTGTCCAAACTCATCAATGTATCTTATCATG
TCTGGATCTCTAGCTTCGTGTCAAGGACGGTGAGGCGCGCCAATATTGGC
TATTGGCCATTGCATACGTTGTATCCATATCATAATATGTACAGCGCGC

FIG. 1 shows a physical map of the thus obtained vector Delta-pEE12.4. Vector Delta-pEE12.4 was used for cloning the short fragment of the mCMV promoter.

Cloning of the Short Fragment of the mCMV Promoter into Vector Delta-Pee12.4

The mCMV-short fragment was amplified by PCR by using the forward primer 3, and the reverse primer 4. As template, mCMV DNA comprising the mCMV promoter (kindly provided by Dr. Clive Sweet, Univ. Birmingham) was used. The scheme of the PCR amplification is outlined in FIG. 2.

The thus obtained PCR fragment (0.5 kb) representing the mCMV-short fragment has the following sequence (SEQ ID No. 11; primer sequences underlined, restriction sites in bold):

CGTATAGGCGCGCCTACTGAGTCATTAGGGACTTTCCAATGGGTTTTGCC
CAGTACATAAGGTCAATAGGGGTGAATCAACAGGAAAGTCCCATTGGAGC
CAAGTACACTGAGTCAATAGGGACTTTCCATTGGGTTTTGCCCAGTACAA
AAGGTCAATAGGGGGTGAGTCAATGGGTTTTTCCCATTATTGGCACGTAC
ATAAGGTCAATAGGGGTGAGTCATTGGGTTTTTCCAGCCAATTTAATTAA
AACGCCATGTACTTTCCCACCATTGACGTCAATGGGCTATTGAAACTAAT
GCAACGTGACCTTTAAACGGTACTTTCCCATAGCTGATTAATGGGAAAGT
ACCGTTCTCGAGCCAATACACGTCAATGGGAAGTGAAAGGGCAGCCAAAA
CGTAACACCGCCCCGGTTTTCCCCTGGAAATTCCATATTGGCACGCATTC
TATTGGCTGAGCTGCGTTCTACGTGGGTATAAGAGGCGCGACCAGCGTCG
GTACCGTCGCAGTCTTCGGTCTGACCACCGTAGAACGCAGAAGCTTCGAT
GC

This fragment shown schematically in FIG. 3 was cloned into vector Delta-pEE12.4, previously cut with Asc I and Hind III. By this, the 5′-UTR and intron A region of the human CMV sequence in the vector were removed.

Cloning of a mCMV-Short Intron a Fragment into Vector Delta-pEE12.4.

The mCMV-short fragment and the human intron A fragment were generated by two separate PCR reactions and then joined together, as follows.

a) Amplification and Cloning of the mCMV-Short Fragment

For amplification of the mCMV-short fragment the forward primer 5 and the reverse primer 6 were used. The scheme of the PCR amplification is outlined in FIG. 2.

The thus obtained PCR product has the following sequence (SEQ ID No. 12; primer sequences underlined, restriction sites in bold):

GATCGGCGCGCCTAAGCTACTGAGTCATTAGGGACTTTCAATGGGTTTTG
CCCAGTACATAAGGTCAATAGGGGTGAATCAACAGGAAAGTCCCATTGGA
GCCAAGTACACTGAGTCAATAGGGACTTTCCATTGGGTTTTGCCCAGTAC
AAAAGGTCAATAGGGGGTGAGTCAATGGGTTTTTCCCATTATTGGCACGT
ACATAAGGTCAATAGGGGTGAGTCATTGGGTTTTTCCAGCCAATTTAATT
AAAACGCCATGTACTTTCCCACCATTGACGTCAATGGGCTATTGAAACTA
ATGCAACGTGACCTTTAAACGGTACTTTCCCATAGCTGATTAATGGGAAA
GTACCGTTCTCGAGCCAATACACGTCAATGGGAAGTGAAAGGGCAGCCAA
AACGTAACACCGCCCCGGTTTTCCCCTGGAAATTCCATATTGGCACGCAT
TCTATTGGCTGAGCTGCGTTCTACGTGGGTATAAGAGGCGCGACCAGCGT
CGGTACCGTCGCAGTCTTCGGTCTGACCACCGTAGAACGCAGCCTCAGGG
ATCGA

This PCR fragment was cloned into the cloning vector pCR4-TOTO (Invitrogen) whereby vector pCR-4-TOTO+mCMV-short was obtained.

### b) Amplification and Cloning of the Human Intron a Fragment

For amplification of the human 5′-UTR-intron A fragment the forward primer 7 and the reverse primer 8 were used. As template DNA, vector pEE12.4 was used.

The PCR product obtained had the following sequence (SEQ ID No. 13; primer sequences underlined, restriction sites in bold):

GATCCCTCAGGACCTCCATAGAAGACACCGGGACCGATCCAGCCTCCGCG
GCCGGGAACGGTGCATTGGAACGCGGATTCCCCGTGCCAAGAGTGACGTA
AGTACCGCCTATAGAGTCTATAGGCCCACCCCCTTGGCTTCTTATGCATG
CTATACTGTTTTTGGCTTGGGGTCTATACACCCCCGCTTCCTCATGTTAT
AGGTGATGGTATAGCTTAGCCTATAGGTGTGGGTTATTGACCATTATTGA
CCACTCCCCTATTGGTGACGATACTTTCCATTACTAATCCATAACATGGC
TCTTTGCCACAACTCTCTTTATTGGCTATATGCCAATACACTGTCCTTCA
GAGACTGACACGGACTCTGTATTTTTACAGGATGGGGTCTCATTTATTAT
TTACAAATTCACATATACAACACCACCGTCCCCAGTGCCCGCAGTTTTTA
TTAAACATAACGTGGGATCTCCACGCGAATCTCGGGTACGTGTTCCGGAC
ATGGGCTCTTCTCCGGTAGCGGCGGAGCTTCTACATCCGAGCCCTGCTCC
CATGCCTCCAGCGACTCATGGTCGCTCGGCAGCTCCTTGCTCCTAACAGT
GGAGGCCAGACTTAGGCACAGCACGATGCCCACCACCACCAGTGTGCCGC
ACAAGGCCGTGGCGGTAGGGTATGTGTCTGAAAATGAGCTCGGGGAGCGG
GCTTGCACCGCTGACGCATTTGGAAGACTTAAGGCAGCGGCAGAAGAAGA
TGCAGGCAGCTGAGTTGTTGTGTTCTGATAAGAGTCAGAGGTAACTCCCG
TTGCGGTGCTGTTAACGGTGGAGGGCAGTGTAGTCTGAGCAGTACTCGTT
GCTGCCGCGCGCGCCACCAGACATAATAGCTGACAGACTAACAGACTGTT
CCTTTCCATGGGTCTTTTCTGCAGTCACCGTCCTTGACACGAAGCTTGA
TC

The thus obtained PCR fragment was cloned into cloning vector pCR4-TOTO (Invitrogen) whereby vector pCR4-TOTO+intron A was obtained.

c) Joining of the mCMV-Short-Intron a Fragment

The mCMV-short PCR product comprising the short mCMV promoter fragment was cut out of vector pCR4-TOTO+mCMVshort with Asc I and Bsu36 I, and cloned into vector pCR4-TOTO+Intron A whereby vector pCR4-TOTO I mCMV-Intron A was obtained. From this vector the fragment mCMV-short-intron A comprising the mCMV promoter and the first human intron A was cut out, using Asc I and Hind III. The structure of this fragment is schematically depicted in FIG. 4. The sequence (SEQ ID No. 14) of this fragment is as follows (primer sequences underlined, restriction sites in bold):

GGCGCGCCTAAGCTACTGAGTCATTAGGGACTTTCAATGGGTTTTGCCCA
GTACATAAGGTCAATAGGGGTGAATCAACAGGAAAGTCCCATTGGAGCCA
AGTACACTGAGTCAATAGGGACTTTCCATTGGGTTTTGCCCAGTACAAAA
GGTCAATAGGGGGTGAGTCAATGGGTTTTTCCCATTATTGGCACGTACAT
AAGGTCAATAGGGGTGAGTCATTGGGTTTTTCCAGCCAATTTAATTAAAA
CGCCATGTACTTTCCCACCATTGACGTCAATGGGCTATTGAAACTAATGC
AACGTGACCTTTAAACGGTACTTTCCCATAGCTGATTAATGGGAAAGTAC
CGTTCTCGAGCCAATACACGTCAATGGGAAGTGAAAGGGCAGCCAAAACG
TAACACCGCCCCGGTTTTCCCCTGGAAATTCCATATTGGCACGCATTCTA
TTGGCTGAGCTGCGTTCTACGTGGGTATAAGAGGCGCGACCAGCGTCGGT
ACCGTCGCAGTCTTCGGTCTGACCACCGTAGAACGCAGCCTCAGGACCTC
CATAGAAGACACCGGGACCGATCCAGCCTCCGCGGCCGGGAACGGTGCAT
TGGAACGCGGATTCCCCGTGCCAAGAGTGACGTAAGTACCGCCTATAGAG
TCTATAGGCCCACCCCCTTGGCTTCTTATGCATGCTATACTGTTTTTGGC
TTGGGGTCTATACACCCCCGCTTCCTCATGTTATAGGTGATGGTATAGCT
TAGCCTATAGGTGTGGGTTATTGACCATTATTGACCACTCCCCTATTGGT
GACGATACTTTCCATTACTAATCCATAACATGGCTCTTTGCCACAACTCT
CTTTATTGGCTATATGCCAATACACTGTCCTTCAGAGACTGACACGGACT
CTGTATTTTTACAGGATGGGGTCTCATTTATTATTTACAAATTCACATAT
ACAACACCACCGTCCCCAGTGCCCGCAGTTTTTATTAAACATAACGTGGG
ATCTCCACGCGAATCTCGGGTACGTGTTCCGGACATGGGCTCTTCTCCGG
TAGCGGCGGAGCTTCTACATCCGAGCCCTGCTCCCATGCCTCCAGCGACT
CATGGTCGCTCGGCAGCTCCTTGCTCCTAACAGTGGAGGCCAGACTTAGG
CACAGCACGATGCCCACCACCACCAGTGTGCCGCACAAGGCCGTGGCGGT
AGGGTATGTGTCTGAAAATGAGCTCGGGGAGCGGGCTTGCACCGCTGACG
CATTTGGAAGACTTAAGGCAGCGGCAGAAGAAGATGCAGGCAGCTGAGTT
GTTGTGTTCTGATAAGAGTCAGAGGTAACTCCCGTTGCGGTGCTGTTAAC
GGTGGAGGGCAGTGTAGTCTGAGCAGTACTCGTTGCTGCCGCGCGCGCCA
CCAGACATAATAGCTGACAGACTAACAGACTGTTCCTTTCCATGGGTCTT
TTCTGCAGTCACCGTCCTTGACACGAAGCTT

This fragment was then cloned into a modified version of the GS-vector pEE12.4, Delta-pEE12.4 vector, as described above, using the Asc I-Hind III sites whereby vector Delta-pEE12.4-mcmv/int was obtained.

Cloning of the Short Fragment of the mCMV Promoter into Vector pEE6.4

The mCMV-short fragment was amplified by PCR by using the forward primer 9, and the reverse primer 4. As template, mCMV DNA comprising the mCMV promoter was used. The scheme of the PCR amplification is outlined in FIG. 2.

The thus obtained PCR product (0.5 kb) representing the mCMV-short fragment has the following sequence (SEQ ID No. 15; primer sequences underlined, restriction sites in bold):

GATCGCTAGCGGCCGCTGAGGCGCGCCTACTGAGTCATTAGGGACTTTCCAATGGGTTTTG CCCAGTACATAAGGTCAATAGGGGTGAATCAACAGGAAAGTCCCATTGGAGCCAAGTACAC TGAGTCAATAGGGACTTTCCATTGGGTTTTGCCCAGTACAAAAGGTCAATAGGGGGTGAGT CAATGGGTTTTTCCCATTATTGGCACGTACATAAGGTCAATAGGGGTGAGTCATTGGGTTT TTCCAGCCAATTTAATTAAAACGCCATGTACTTTCCCACCATTGACGTCAATGGGCTATTG AAACTAATGCAACGTGACCTTTAAACGGTACTTTCCCATAGCTGATTAATGGGAAAGTACC GTTCTCGAGCCAATACACGTCAATGGGAAGTGAAAGGGCAGCCAAAACGTAACACCGCCCC GGTTTTCCCCTGGAAATTCCATATTGGCACGCATTCTATTGGCTGAGCTGCGTTCTACGTG GGTATAAGAGGCGCGACCAGCGTCGGTACCGTCGCAGTCTTCGGTCTGACCACCGTAGAAC GCAGAAGCTTCGATGC

This fragment is very similar to that of FIG. 3, except with a Not I site in addition to the Asc I site. The fragment was cloned into vector pEE6.4, previously cut with Asc I and Hind III. This generated the vector pEE6.4-mCMVshort.

Cloning of a mCMV-Short Intron a Fragment into Vector pEE6.4.

The mCMV-short fragment and the human intron A fragment were generated by two separate PCR reactions and then joined together, as follows.

a) Amplification and Cloning of the mCMV-Short Fragment

For amplification of the mCMV-short fragment the forward primer 9 and the reverse primer 6 were used (Table 1). The scheme of the PCR amplification is outlined in FIG. 2.

The thus obtained PCR product (mCMVshort-pEE6.4) has the following sequence (SEQ ID No. 16; primer sequences underlined, restriction sites in bold):

GATCGCTAGCGGCCGCTGAGGCGCGCCTACTGAGTCATTAGGGACTTTCA
ATGGGTTTTGCCCAGTACATAAGGTCAATAGGGGTGAATCAACAGGAAAG
TCCCATTGGAGCCAAGTACACTGAGTCAATAGGGACTTTCCATTGGGTTT
TGCCCAGTACAAAAGGTCAATAGGGGGTGAGTCAATGGGTTTTTCCCATT
ATTGGCACGTACATAAGGTCAATAGGGGTGAGTCATTGGGTTTTTCCAGC
CAATTTAATTAAAACGCCATGTACTTTCCCACCATTGACGTCAATGGGCT
ATTGAAACTAATGCAACGTGACCTTTAAACGGTACTTTCCCATAGCTGAT
TAATGGGAAAGTACCGTTCTCGAGCCAATACACGTCAATGGGAAGTGAAA
GGGCAGCCAAAACGTAACACCGCCCCGGTTTTCCCCTGGAAATTCCATAT
TGGCACGCATTCTATTGGCTGAGCTGCGTTCTACGTGGGTATAAGAGGCG
CGACCAGCGTCGGTACCGTCGCAGTCTTCGGTCTGACCACCGTAGAACGC
AGCCTCAGGGATCGA

This PCR fragment was cloned into the cloning vector pCR4-TOPO+intron A whereby vector pCR4-TOTO-mCMVshort(pEE6.4) was obtained.

b) Joining of the mCMV(pEE6.4)-Short-Intron a Fragment

The mCMV-short(pEE6.4) PCR product comprising the short mCMV promoter fragment was cut out of vector pCR4-TOTO-mCMVshort(pEE6.4) with Asc I and Bsu36 I, and cloned into vector pCR4-TOTO+Intron A whereby vector pCR4-TOTO 1 mCMV(pEE6.4)-Intron A was obtained. From this vector the fragment mCMV-short(pEE6.4)-intron A, comprising the mCMV promoter and the first human intron A, was cut out using Not I and Hind III. The structure of this fragment is identical as that schematically depicted in FIG. 4 except that there is a newly introduced Not I site at the 5′ end. The sequence of this fragment (SEQ ID No. 17) is as follows (primer sequences underlined, restriction sites in bold):

GCGGCCGCTGAGGCGCGCCTACTGAGTCATTAGGGACTTTCAATGGGTTT
TGCCCAGTACATAAGGTCAATAGGGGTGAATCAACAGGAAAGTCCCATTG
GAGCCAAGTACACTGAGTCAATAGGGACTTTCCATTGGGTTTTGCCCAGT
ACAAAAGGTCAATAGGGGGTGAGTCAATGGGTTTTTCCCATTATTGGCAC
GTACATAAGGTCAATAGGGGTGAGTCATTGGGTTTTTCCAGCCAATTTAA
TTAAAACGCCATGTACTTTCCCACCATTGACGTCAATGGGCTATTGAAAC
TAATGCAACGTGACCTTTAAACGGTACTTTCCCATAGCTGATTAATGGGA
AAGTACCGTTCTCGAGCCAATACACGTCAATGGGAAGTGAAAGGGCAGCC
AAAACGTAACACCGCCCCGGTTTTCCCCTGGAAATTCCATATTGGCACGC
ATTCTATTGGCTGAGCTGCGTTCTACGTGGGTATAAGAGGCGCGACCAGC
GTCGGTACCGTCGCAGTCTTCGGTCTGACCACCGTAGAACGCAGCCTCAG
GACCTCCATAGAAGACACCGGGACCGATCCAGCCTCCGCGGCCGGGAACG
GTGCATTGGAACGCGGATTCCCCGTGCCAAGAGTGACGTAAGTACCGCCT
ATAGAGTCTATAGGCCCACCCCCTTGGCTTCTTATGCATGCTATACTGTT
TTTGGCTTGGGGTCTATACACCCCCGCTTCCTCATGTTATAGGTGATGGT
ATAGCTTAGCCTATAGGTGTGGGTTATTGACCATTATTGACCACTCCCCT
ATTGGTGACGATACTTTCCATTACTAATCCATAACATGGCTCTTTGCCAC
AACTCTCTTTATTGGCTATATGCCAATACACTGTCCTTCAGAGACTGACA
CGGACTCTGTATTTTTACAGGATGGGGTCTCATTTATTATTTACAAATTC
ACATATACAACACCACCGTCCCCAGTGCCCGCAGTTTTTATTAAACATAA
CGTGGGATCTCCACGCGAATCTCGGGTACGTGTTCCGGACATGGGCTCTT
CTCCGGTAGCGGCGGAGCTTCTACATCCGAGCCCTGCTCCCATGCCTCCA
GCGACTCATGGTCGCTCGGCAGCTCCTTGCTCCTAACAGTGGAGGCCAGA
CTTAGGCACAGCACGATGCCCACCACCACCAGTGTGCCGCACAAGGCCGT
GGCGGTAGGGTATGTGTCTGAAAATGAGCTCGGGGAGCGGGCTTGCACCG
CTGACGCATTTGGAAGACTTAAGGCAGCGGCAGAAGAAGATGCAGGCAGC
TGAGTTGTTGTGTTCTGATAAGAGTCAGAGGTAACTCCCGTTGCGGTGCT
GTTAACGGTGGAGGGCAGTGTAGTCTGAGCAGTACTCGTTGCTGCCGCGC
GCGCCACCAGACATAATAGCTGACAGACTAACAGACTGTTCCTTTCCATG
GGTCTTTTCTGCAGTCACCGTCCTTGACACGAAGCTT

### Protein Expression Studies

The goal of the antibody expression studies was to compare the expression of an IgG4/kappa antibody in CHOK1SV cells under the control of either the mCMV promoter or the hCMV promoter.

Therefore, double gene vectors for the expression of the IgG4/kappa antibody cB72.3 were constructed each of which contained both the heavy chain gene and the light chain gene for the IgG4/kappa antibody wherein each gene was separately under the control of the same regulatory unit.

The entire regions coding for heavy chain and for light chain of antibody cB72.3 were, separately, cloned out of existing vectors with Hind III and EcoR I and cloned into the vectors generated above: light chain into (i) Delta-pEE12.4+mCMV and (ii) Delta-pEE12.4/mCMV+intron A, and the heavy chain coding fragment into (i) pEE6.4-mCMV and (ii) pEE6.4-mCMV+intron A.

Double gene vectors were generated by digestion of all vectors with Not I and Pvu I, and cloning the appropriate fragments (containing the CMV promoters and antibody-chain-coding sequences) together, such that two double gene vectors were generated, namely vector pcB72.3-mCMV (FIG. 5) containing the suitable fragments from Delta-pEE12.4+mCMV and pEE6.4-mCMV, and vector pcB72.3-mCMV+intron A (FIG. 6), containing the suitable fragments from Delta-pEE12.4/mCMV+intron A and pEE6.4-mCMV+intron A. In vector pcB72.3-mCMV the antibody expression is driven by the mCMV promoter. In vector pcB72.3-mCMV+intron A the antibody expression is driven by the mCMV promoter plus the first human CMV intron.

The control vector, pcB72.3 (FIG. 7) contains the same heavy chain and light chain coding regions under the regulation of the human CMV promoter plus human intron A.

These constructs were introduced into CHO cells by stable transfection and the antibody expression was studied.

The results of these experiments are shown in FIG. 8. FIG. 8 shows that the expression level of the IgG4/kappa antibody in stably transfected CHO-K1SV cells from the regulatory unit consisting of the mCMV promoter and the hCMV intron is much higher than the expression from the short mCMV promoter alone (statistically significant at p<0.0001). Further, it corresponds to the antibody expression level from the hCMV promoter plus the hCMV intron. Thus, expression of a heterologous protein from the regulatory unit consisting of the mCMV promoter and the hCMV intron is at least as good as expression from hCMV promoter and hCMV intron A.

1. A mammalian expression vector comprising the murine CMV promoter and the first human CMV intron operably linked to a heterologous coding sequence.

2. The mammalian expression vector according to claim 1, wherein the murine CMV promoter includes the mCMV promoter sequence from position −491 to position +36.

3. The mammalian expression vector according to claim 1, wherein the murine CMV promoter includes the mCMV promoter sequence from position −1336 to position +36.

4. The mammalian expression vector according to claim 3, wherein the vector comprises a second transcription unit encoding a selectable marker.

5. A mammalian host cell containing a mammalian expression vector according claim 4.

6. The mammalian host cell according to claim 5 wherein the cell is a Chinese Hamster Ovary (CHO) cell.

7. A process for the production of recombinant protein, comprising the steps of:
a) transfecting a mammalian host cell with an expression vector comprising a murine CMV promoter and the first human CMV intron operably linked to a sequence coding for the recombinant protein;
b) culturing the host cell under appropriate conditions to enable propagation of the cell and expression of the recombinant protein; and
c) harvesting the recombinant protein produced.

8. The process according to claim 7, wherein the mammalian host cell is a CHO cell.

9. A process comprising utilizing first human CMV intron to enhance the activity of murine CMV promoter for expressing a recombinant protein.

10. The mammalian expression vector according to claim 1, wherein the vector comprises a second transcription unit encoding a selectable marker.

11. A mammalian host cell containing a mammalian expression vector according to claim 1.

12. The mammalian host cell according to claim 11, wherein the cell is a Chinese Hamster Ovary (CHO) cell.

13. The mammalian expression vector according to claim 2, wherein the vector comprises a second transcription unit encoding a selectable marker.

14. The mammalian host cell containing a mammalian expression vector according to claim 2.

15. The mammalian host cell according to claim 14, wherein the cell is a Chinese Hamster Ovary (CHO) cell.

16. The mammalian expression vector according to claim 1, wherein the vector comprises a second transcription encoding a glutamine synthetase (GS) marker.

17. The mammalian expression vector according to claim 2, wherein the vector comprises a second transcription unit encoding a glutamine synthetase (GS) marker.

18. The mammalian expression vector according to claim 3, wherein the vector comprises a second transcription unit encoding glutamine synthetase (GS) marker.
